(19) United States
(12) Patent Application Publication
(10) Pub. No.: US 2025/0043099 A1
(43) Pub. Date: Feb. 6, 2025

(54) DIGESTION OF POLY(ETHYLENE TEREPHTHALATE) USING LONG CHAIN ALCOHOLS

(71) Applicants: Matthew Green, Phoenix, AZ (US); Elham Fini, Phoenix, AZ (US); Albert Hung, Tempe, AZ (US); Robert David Allen, Golden, CO (US)

(72) Inventors: Matthew Green, Phoenix, AZ (US); Elham Fini, Phoenix, AZ (US); Albert Hung, Tempe, AZ (US); Robert David Allen, Golden, CO (US)

(21) Appl. No.: 18/792,229

(22) Filed: Aug. 1, 2024

Related U.S. Application Data
(60) Provisional application No. 63/517,157, filed on Aug. 2, 2023.

(51) Int. Cl. C08J 11/24; C08G 63/183; C08K 5/20; C08L 23/06; C08L 95/00

(52) U.S. Cl. CPC C08J 11/24; C08G 63/183; C08K 5/20; C08L 23/06; C08L 95/00; C08J 2367/03; C08L 2207/20; C08L 2555/34; C08L 2555/86

(57) ABSTRACT

Digesting poly(ethylene terephthalate) includes combining poly(ethylene terephthalate) with a solvent that includes one or more long chain alcohols to yield a reaction mixture, and heating the reaction mixture to a temperature between about 20° C. and about 300° C. to yield a product including one or more alkyl terephthalates. A modified asphalt includes asphalt, an asphalt binder, molten polyolefin, and the one or more alkyl terephthalates

### CROSS-REFERENCE TO RELATED APPLICATION

This application claims the benefit of U.S. Patent Application No. 63/517,157 filed on Aug. 2, 2023, which is incorporated by reference herein in its entirety.

### STATEMENT OF GOVERNMENT INTEREST

This invention was made with government support under 1935723 awarded by the National Science Foundation. The government has certain rights in the invention.

### TECHNICAL FIELD

This invention relates to digestion of poly(ethylene terephthalate) using long chain alcohols to yield alkyl terephthalamides and alkyl terephthalates, which are subsequently combined with molten low-density polyethylene to form a binder additive for asphalt.

### BACKGROUND

Poly(ethylene terephthalate) (PET) is a commodity plastic used in a variety of single-use applications (to-go containers, food packaging, water bottles, etc.). The accumulation of plastic waste such as PET in landfills and the environment is fueling a push to advance polymer recycling technology. Chemically recycling plastics to break down the macromolecules into monomers and oligomers is particularly attractive as it enables a truly circular plastic economy.

### SUMMARY

This disclosure describes production of an asphalt binder made from waste poly(ethylene terephthalate) (PET). Waste PET is digested into alkyl terephthalamides and alkyl terephthalates using long chain alcohols in a one-pot reaction. The alkyl terephthalamides and terephthalates are combined with molten low-density polyethylene (LDPE) to form a PET-based binder additive. This PET-based binder additive is combined with bitumen or other asphalt binder yield a modified binder. The modified binder is combined with aggregate to form asphalt suitable for use in the construction of roadways, roofing shingles, floor tiles, and other products.

In a first general aspect, digesting poly(ethylene terephthalate) includes combining poly(ethylene terephthalate) with a solvent that includes one or more long chain alcohols to yield a reaction mixture, and heating the reaction mixture to a temperature between about 20° C. and about 300° C. to yield a product including one or more alkyl terephthalates.

Implementations may include one or more of the following features. The poly(ethylene terephthalate) and the long chain alcohol can be combined in a ratio such that a number of ester linkages in the poly(ethylene terephthalate) to a number of moles of the long chain alcohol about 2:1 or greater. The poly(ethylene terephthalate) and the long chain alcohol can be combined in a ratio such that a number of ester linkages in the poly(ethylene terephthalate) to a number of moles of the long chain alcohol is about 10:1 or less. A melting point of the one or more alkyl terephthalates exceeds the melting point of the one or more long chain alcohols. The one or more long chain alcohols may include a C6-C20 alcohol.

The poly(ethylene terephthalate) can be particulate or in the form of flakes. The method may include combining the one or more alkyl terephthalates with molten polyolefin to yield a mixture. The molten polyolefin may include molten low density polyethylene, molten high density polyethylene, molten linear low density polyethylene, molten polypropylene, or any combination thereof. The method may include combining the mixture with an asphalt binder to yield a composite. The method may include combining the composite with asphalt to yield a modified asphalt. The asphalt binder may include bitumen.

The product further may include one or more alkyl terephthalamides. The one or more alkyl terephthalates are represented by the following structure:

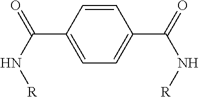

where each r is independently a C6-C20 alkyl or C6-C20 carboxyalkyl, and at least one r is a C6-C20 alkyl. The one or more long chain alcohols may include octanol, dodecanol, hexadecanol, or a combination thereof. The solvent further may include one or more long chain alkylamines. The one or more long chain alkylamines may include octylamine, dodecylamine, hexadecylamine, or a combination thereof. The one or more long chain alcohols react with the poly(ethylene terephthalate) to yield the one or more alkyl terephthalates.

In a second general aspect, a modified asphalt includes asphalt, an asphalt binder; molten polyolefin; and the one or more alkyl terephthalates prepared by the method of claim 1.c

The details of one or more embodiments of the subject matter of this disclosure are set forth in the accompanying drawings and the description. Other features, aspects, and advantages of the subject matter will become apparent from the description, the drawings, and the claims.

### BRIEF DESCRIPTION OF DRAWINGS

FIG. 1 shows steps in the production and study of an asphalt modifier made by a process in which poly(ethylene terephthalate) is digested and used to dissolve low density polyethylene.

FIG. 2 is a schematic of a parallel-plate setup.

FIG. 3 shows measurements of zero shear viscosity, shear thinning, and the onset of shear thinning from a plot of viscosity versus shear rate.

### DETAILED DESCRIPTION

This disclosure describes mixed waste plastics compatibilizers for asphalt, composites including the mixed waste plastics compatibilizers, modified asphalts including the composites, and methods of preparing the compatibilizers, composites, and plastic-modified asphalts. The compatibilizers are formed from waste poly(ethylene terephthalate) (PET), and promote dispersion of polyolefin particles (e.g., low-density polyethylene (LDPE), high-density polyethylene (HDPE), linear low-density polyethylene (LLDPE), polypropylene (PP), or any combination or copolymer thereof) in bitumen. The composites include polyolefin particles dispersed in the bitumen with the PET compatibilizer. Asphalt is combined with the composite to yield the modified asphalts.

Preparation of the compatibilizer includes combining PET with a long chain alcohol reactant to digest the PET. The monomers and oligomers of the digested PET are combined with molten polyolefin (e.g., LDPE) to yield a composite. Crystalline PET particles present in the digested PET impart additional strength to the composite. The properties of the composite can be tailored by selecting a hydrocarbon chain length of the polyolefin to avoid segregation and enhance asphalt healing capacity and resistance to aging. Thus, use of one waste stream (PET) to compatibilize another waste stream (e.g., polyolefin(s)) can be advantageous for a variety of reasons.

This disclosure describes a process to turn waste plastics into modifiers for asphalt pavements to extend pavement service life. Poly(ethylene terephthalate) (PET) is digested to create a terephthalamide compatibilizer. Digesting the PET includes combining waste PET with a long chain alcohol reactant and depolymerizing the PET to yield alkyl terephthalate and alkyl terephthalamide monomers and oligomers. In some implementations, digesting the PET includes melting the PET with the long chain alcohol reactant to yield a mixture, and feeding the mixture through an extruder for continuous or batch production of a compatibilizer that includes terephthalates and terephthalamides. The molten PET (e.g., T>˜240° C.) is free or substantially free of crystallinity and promotes fast digestion kinetics. In some implementations, the long chain alcohol reactant plasticizes the PET at moderate temperatures (100° C.<T<200° C.), thereby reducing viscosity. This yields a semi-crystalline PET that is partially reacted due to relatively fast digestion kinetics of the amorphous component, and results in compatibilizer that includes a mixture of terephthalates, terephthalamides, and crystalline PET. Terephthalates resulting from these implementations have better hydrolytic stability than ester-based phthalates.

Suitable reaction temperatures are from about 20° C. to about 300° C. The long chain alcohols are represented as R—OH, where R is a linear or branched C6-C20 alkyl or linear or branched C6-20 carboxyalkyl (e.g., C8-C16, or any combination of C8, C9, C10, C11, C12, C13, C14, C15, and C16, such as C8, C12, and C16). The carboxyl groups of the PET oligomer are terminated with alkyl chains using long chain alcohol. The temperature at which the PET is digested, or processing temperature, is typically between the melting point and the boiling point of the long chain alcohol. To perform the digestion, PET particles (e.g., flakes) are combined with the long chain alcohol at a temperature in a range between about 20° C. and about 300° C.

The stoichiometry (ratio of PET:long chain alcohol) and temperature can be selected based at least in part on the long chain alcohol to control the reaction kinetics as well as the yield and purity of the resulting alkyl terephthalates and alkyl terephthalamides. In some implementations, the poly(ethylene terephthalate) and the long chain alcohol are combined in a ratio such that a number of ester linkages in the poly(ethylene terephthalate) to a number of moles of the long chain alcohol is 2:1 or greater. The poly(ethylene terephthalate) and the long chain alcohol can be combined in a ratio such that a number of ester linkages in the poly(ethylene terephthalate) to a number of moles of the long chain alcohol is about 10:1 or less. There are typically two ester linkages in each repeat unit of the poly(ethylene terephthalate). The reaction progress can be monitored as a function of time using one or more of ¹H NMR spectroscopy, mass spectrometry, melt index or rheological techniques, size exclusion chromatography. The resulting alkyl terephthalamides are shown below, where each R is independently a linear or branched C6-C20 alkyl or a linear or branched C6-C20 carboxyalkyl, and at least one R is a C6-C20 alkyl. That is, the alkyl terephthalamide can be a bis(alkyl) terephthalamide or a mono(alkyl) terephthalamide.

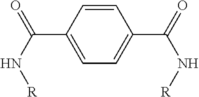

These alkyl terephthalamides have a higher melting point than the corresponding long chain alcohol precursors, allowing for easy separation by hot filtration to remove the precursor(s).

A composite is formed by the combination of the alkyl terephthalates, alkyl terephthalamides, and polyolefin particles with bitumen. The polyolefin particles can include low-density polyethylene (LDPE), high-density polyethylene (HDPE), linear low-density polyethylene (LLDPE), or a copolymer or combination thereof. A largest dimension of the polyolefin particles is typically in a range between about 1 mm and about 100 cm. In some cases, a largest dimension of the polyolefin particles is in a range between about 1 mm and about 10 mm. The processing temperature is typically in a range between about 30° C. to about 80° C. greater than the melting temperature of the polyolefin particles (e.g., between about 150° C. and about 200° C.). The composite typically includes up to about 20 wt % of the polyolefin particles, and up to about 10 wt % of the alkyl terephthalates and alkyl terephthalamides. The alkyl terephthalamides are amphiphilic, with a polar terephthalamide head group 206 and a nonpolar alkyl tail 208, and can serve as compatibilizers to disperse the polyolefin particles in the bitumen. That is, the C6-C20 hydrocarbon chains on the alkyl terephthalamide are nonpolar, and can preferentially orient toward the polyolefin particles, while the polar portion of the terephthalamide can preferentially orient toward the bitumen. This preferential arrangement can help disperse the polyolefin particles in the bitumen to yield a composite.

The composite can be combined with asphalt as an asphalt modifier to yield a modified asphalt. The asphalt can be combined with the asphalt modifier at a temperature in a range between about 150° C. to about 175° C. (e.g., “hot mix”) or less than about 100° C. (e.g., “warm mix”). The temperature is typically selected to be below the melting point of the polyolefin (e.g., below about 100° C. for LDPE or LLDPE or below about 180° C. for HDPE).

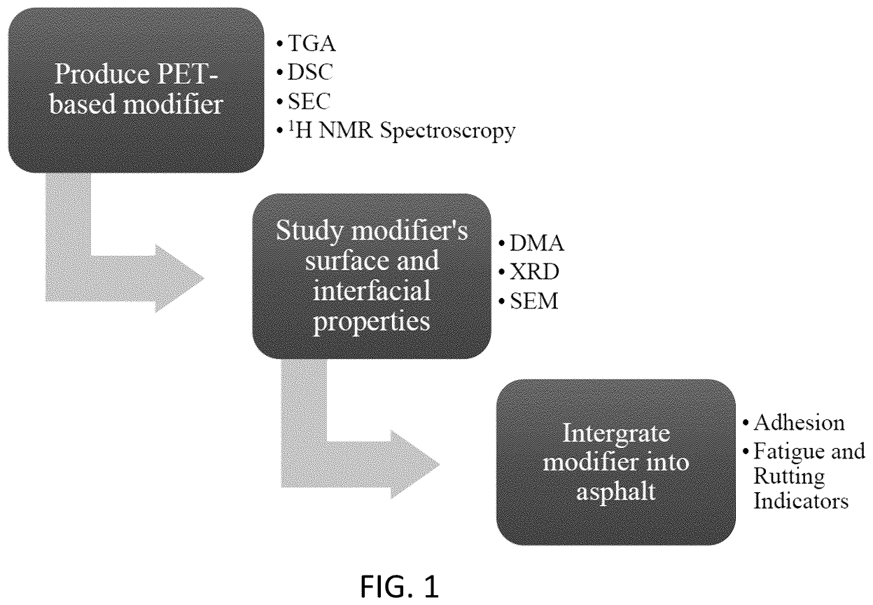

FIG. 1 shows steps in the production and study of an asphalt modifier made by a process in which PET is digested with one or more long chain alcohols and used to dissolve low density polyethylene. In some examples, PET flakes are fed to reactors or extruders along with the long chain alcohols and at sufficient temperatures and pressures to depolymerize the PET to monomers and oligomers. The resulting alkyl terephthalates can be separated via crystallization, and the polar compounds can be used as modifiers to compatibilize other waste plastics (e.g., low density polyethylene, LDPE) in bitumen and asphalt. Examples of suitable alcohols include octanol, dodecanol, and hexadecanol. In some cases, the long chain alcohol(s) is combined with one or more long chain alkylamines. Examples of suitable long chain alkylamines include octylamine, dodecylamine, and hexadecylamine. The long chain alkylamines and alcohols serve as the reaction solvent and reagent in the low temperature digestion of PET, promoting dissolution of the PET and, at sufficient temperature and pressure, initiating reactions to depolymerize PET and form alkyl terephthalates and alkyl terephthalamides.

The resulting alkyl terephthalates and alkyl terephthalamides and have a higher melting point than the long chain alcohols and alkylamines, allowing for easy separation by hot filtration. The resulting terephthalamides and terephthalates can be characterized using a variety of techniques including SEC, differential scanning calorimetry, thermogravimetric analysis, mass spectroscopy, and ¹H NMR spectroscopy.

The surface tension of bitumen with the terephthalamides and terephthalates can be measured to characterize the mixture compatibility. Solutions can be prepared in solvents (e.g., toluene and xylene). Next, the terephthalamides and terephthalates can be mixed at various weight fractions (1-25 wt %) into molten LDPE (mp˜115° C.) to determine solubility. The terephthalamide-loaded and terephthalate-loaded LDPEs can be characterized using differential scanning calorimetry, X-ray diffraction, melt rheology, dynamic mechanical analysis, and scanning electron and transmission electron microscopy to study crystallinity, viscosity, modulus, and microstructure.

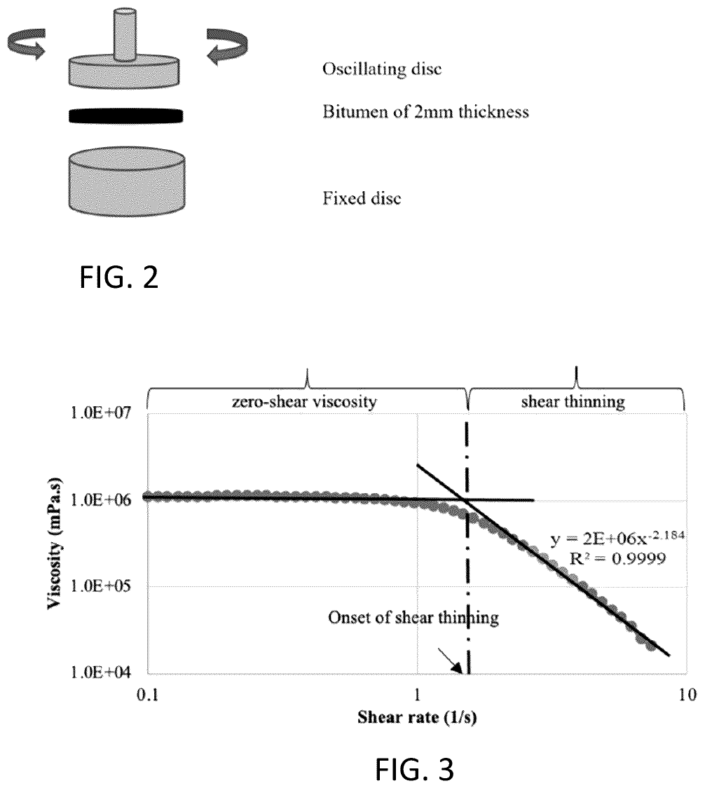

The PET-based modifier can be introduced to asphalt binder (e.g., PG70-10) at 0, 5, 6, and 9% by weight of asphalt to enhance the properties of asphalt. The terephthalamides and terephthalates can be added to bitumen to observe changes in viscosity, homogeneity, and other performance attributes that affect the properties of asphalt. The upper solubility limits of the terephthalamides and terephthalates in asphalt can be assessed. At the binder level, chemical and rheological characteristics before and after short-term and accelerated aging (RTFO and PAV) can be investigated to study, fatigue, rutting, low temperature cracking, and adhesion properties. Moisture susceptibility of the modified bitumen can be evaluated using the moisture-induced shear thinning index (MISTI) test to examine the durability of modified asphalt. To do the MISTI test, a shear ramp test will be performed on asphalt and glass beads (2:1 ratio of modified asphalt and glass beads) representing siliceous inclusions of composite using a dynamic shear rheometer (DSR). Each specimen is then demolded and placed in a parallel-plate test setup of a DSR to be tested, as depicted in FIG. 2. A ramping shear is applied on the specimen, and the viscosity value is measured at each shear rate. A plot of viscosity versus shear rate is then used for analysis. A plot of typical data and the analysis are shown in FIG. 3. The effect of the type and concentration of PET-based modifiers can be assessed to determine the best performing asphalt. Adhesion tests can be run on the optimal blends. Based on the comparison between the chemical components and the physical performances, a preliminary technoeconomic analysis will provide guidance for selecting the type and content of PET-based modifier and projected cost and longevity.

Although this disclosure contains many specific embodiment details, these should not be construed as limitations on the scope of the subject matter or on the scope of what may be claimed, but rather as descriptions of features that may be specific to particular embodiments. Certain features that are described in this disclosure in the context of separate embodiments can also be implemented, in combination, in a single embodiment. Conversely, various features that are described in the context of a single embodiment can also be implemented in multiple embodiments, separately, or in any suitable sub-combination. Moreover, although previously described features may be described as acting in certain combinations and even initially claimed as such, one or more features from a claimed combination can, in some cases, be excised from the combination, and the claimed combination may be directed to a sub-combination or variation of a sub-combination.

Particular embodiments of the subject matter have been described. Other embodiments, alterations, and permutations of the described embodiments are within the scope of the following claims as will be apparent to those skilled in the art. While operations are depicted in the drawings or claims in a particular order, this should not be understood as requiring that such operations be performed in the particular order shown or in sequential order, or that all illustrated operations be performed (some operations may be considered optional), to achieve desirable results.

Accordingly, the previously described example embodiments do not define or constrain this disclosure. Other changes, substitutions, and alterations are also possible without departing from the spirit and scope of this disclosure.

What is claimed is:

1. A method of digesting poly(ethylene terephthalate), the method comprising:
combining poly(ethylene terephthalate) with a solvent comprising one or more long chain alcohols to yield a reaction mixture; and
heating the reaction mixture to a temperature between about 20° C. and about 300° C. to yield a product comprising one or more alkyl terephthalates.

2. The method of claim 1, wherein the poly(ethylene terephthalate) and the long chain alcohol are combined in a ratio such that a number of ester linkages in the poly(ethylene terephthalate) to a number of moles of the long chain alcohol is about 2:1 or greater.

3. The method of claim 2, wherein the poly(ethylene terephthalate) and the long chain alcohol are combined in a ratio such that the number of ester linkages in the poly(ethylene terephthalate) to the number of moles of the long chain alcohol is about 10:1 or less.

4. The method of claim 1, further comprising selecting the temperature to control the yield and purity of the one or more alkyl terephthalates.

5. The method of claim 1, wherein a melting point of the one or more alkyl terephthalates exceeds the melting point of the one or more long chain alcohols.

6. The method of claim 1, wherein the one or more long chain alcohols comprise a C6-C20 alcohol.

7. The method of claim 1, wherein the poly(ethylene terephthalate) is particulate.

8. The method of claim 7, wherein the poly(ethylene terephthalate) is in the form of flakes.

9. The method of claim 1, further comprising combining the one or more alkyl terephthalates with molten polyolefin to yield a mixture.

10. The method of claim 9, wherein the molten polyolefin comprises molten low density polyethylene, molten high density polyethylene, molten linear low density polyethylene, molten polypropylene, or any combination thereof.

11. The method of claim 9, further comprising combining the mixture with an asphalt binder to yield a composite.

12. The method of claim 11, further comprising combining the composite with asphalt to yield a modified asphalt.

13. The method of claim 11, wherein the asphalt binder comprises bitumen.

14. The method of claim 1, wherein the product further comprises one or more alkyl terephthalamides.

15. The method of claim 14, wherein the one or more alkyl terephthalates are represented by the following structure: 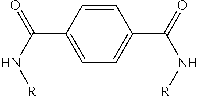
wherein each R is independently a C6-C20 alkyl or C6-C20 carboxyalkyl, and at least one R is a C6-C20 alkyl.

16. The method of claim 1, wherein the one or more long chain alcohols comprise octanol, dodecanol, hexadecanol, or a combination thereof.

17. The method of claim 1, wherein the solvent further comprises one or more long chain alkylamines.

18. The method of claim 17, wherein the one or more long chain alkylamines comprise octylamine, dodecylamine, hexadecylamine, or a combination thereof.

19. The method of claim 1, wherein the one or more long chain alcohols react with the poly(ethylene terephthalate) to yield the one or more alkyl terephthalates.

20. A modified asphalt comprising:
asphalt;
an asphalt binder,
molten polyolefin; and
the one or more alkyl terephthalates prepared by the method of claim 1.
